(19) United States
(12) Patent Application Publication
(10) Pub. No.: US 2006/0058345 A1
(43) Pub. Date: Mar. 16, 2006

(54) Use of papaverine-like vasodilator and pharmaceutical composition

(75) Inventors: Dorothea Gross, St. Ingbert (DE); Frank Holzer, St. Ingbert (DE)

Correspondence Address: SYNNESTVEDT & LECHNER, LLP, 2600 ARAMARK TOWER, 1101 MARKET STREET, PHILADELPHIA, PA, 191072950, US

(73) Assignee: Ursapharm Arzneimittel GmbH & Co KG, Saarbrucken (DE)

(21) Appl. No.: 10/523,864

(22) PCT Filed: Aug. 8, 2003
(86) PCT No.: PCT/EP03/08812

§ 371 Date: Feb. 7, 2005

(30) Foreign Application Priority Data
Aug. 8, 2002 (DE) 102 36 564.4

(51) Int. Cl. A61K 31/47

(52) U.S. Cl. 514/309; 514/308

(57) ABSTRACT

The invention concerns the use of papaverine-like vasodilator for the production of a pharmaceutical composition for the treatment of ophthalmological dysfunctions which are linked to circulatory disturbances of the eye or which are to be attributed to circulatory disturbances of the eye, wherein the pharmaceutical composition is to be applied topically to the eye. The invention further concerns a pharmaceutical composition which includes papaverine-like vasodilator and pharmacologically compatible viscosity regulator.

Many eye diseases are linked to or are to be attributed to blood circulatory disturbances. Such circulatory disturbances can for example result in functional disturbances to the optic nerves (normal pressure glaucoma) and the retina. That can involve on the one hand an inadequate supply of nutrients to the eye and accordingly a chronic nutritional disturbance to the ocular tissue. Of greater significance however is the inadequate supply of oxygen to the eye, caused by the circulatory disturbances in the flow of blood, that is to say hypoxia of the eye.

In the case of a chronic reduced supply of blood to the eye, particularly in the case of vascular optic nerve atrophy, in the case of advanced glaucoma (without high pressure) and the in the case of macular degeneration, it is difficult to express recommendations for a therapy procedure.

As a therapy approach, it would be possible to try to increase the pressure gradient of the flow of blood in the vessels of the eye in order to implement improved circulation. Such a procedure can however result in vessel damage in the long term.

For permanent treatment at the present time in particular thrombocyte aggregation-inhibitors such as acetylsalicylic acid and drugs which improve erythrocyte deformability are prescribed.

In order generally to improve the circulation in blood vessels vasodilators can be systemically administered. It has been found however that a long-term improvement in circulation in the optic nerve and the retina cannot be achieved by systemic administration of vasodilators in the case of a normotonic, in contrast to a hypertonic. In addition there are test results which show that a therapeutically increased circulation in other regions of the body can result in a reduced circulation in the region of the eye.

A syndrome in which circulatory disturbance of the eye can occur is diabetes mellitus. Diabetes mellitus can involve diabetic retinopathy. As it progresses further diabetic retinopathy can result in loss of sight on the part of the diabetic. In the course of the emergence of diabetic retinopathy the situation involves reduced blood circulation and an inadequate supply of oxygen to the retina. As a consequence of the developing retinal hypoxia and a loss of pericytes (adventitia cells) proliferation of endothelium cells occurs, with the formation of microaneurysms and vessel re-formations. In that case intraretinal microvascular anomalies occur. As a consequence thereof the reduced blood circulation is intensified by the formation of a vessel-constricting factor (EDCF) due to endothelium cells in the hypoxic retina.

There is accordingly a need for a pharmaceutical composition for the therapy of diabetic retinopathy.

In the case of circulatory disturbances of the eye such as for example in the case of the above-mentioned diabetic retinopathy, after central vein thrombosis or stenosis of the aorta carotis, the situation can involve vessel re-formation and accordingly neovascularisation glaucoma or hemorrhagic glaucoma.

The object of the present invention is to provide an active substance group or an active substance for the production of a pharmaceutical composition for the therapy of ophthalmological dysfunctions which are linked to circulatory disturbances at the eye or are to be attributed to circulatory disturbances of the eye.

A further object of the present invention is to provide a pharmaceutical composition which can be used in relation to circulatory disturbances of the eye.

The object of the invention is attained by the use of papaverine-like vasodilator for the production of a pharmaceutical composition for the treatment of ophthalmological dysfunctions which are linked to circulatory disturbances of the eye or which are to be attributed to circulatory disturbances of the eye, wherein the pharmaceutical composition is to be applied topically to the eye.

Preferred developments of the use are recited in claims 2 to 6.

The object of the invention is further attained by a pharmaceutical composition which includes papaverine-like vasodilator and pharmacologically compatible viscosity regulator, wherein the papaverine-like vasodilator is selected from the group which consists of ethaverine, moxaverine, elziverine, their pharmacologically compatible salts and mixtures thereof.

Preferred developments of the pharmaceutical composition are recited in claims 8 and 9.

The inventors surprisingly found that papaverine-like vasodilators are suitable for topical application to the eye. The papaverine-like vasodilators are also referred to as musculotropic spasmolytics. The papaverine-like vasodilators cause relaxation of the musculature by direct action on the smooth muscle cells. In accordance with the invention the papaverine-like vasodilators are interpreted as including isoquinoline derivatives which as a common structural element have an isoquinoline ring system and exhibit a vasodilatory action.

The papaverine-like vasodilators exhibit in vitro relaxing effects on smooth-muscular organs including arterial vessels. The actions of the papaverine-like vasodilators are attributed to inhibition of phosphodiesterases (PDE) and a resulting rise in cyclic AMP in muscle cells.

In accordance with a preferred development the papaverine-like vasodilator is selected from the group which consists of papaverine, ethaverine, moxaverine, elziverine, their pharmacologically compatible salts and mixtures thereof. The structural formulae of the above-indicated compounds are set forth hereinafter:

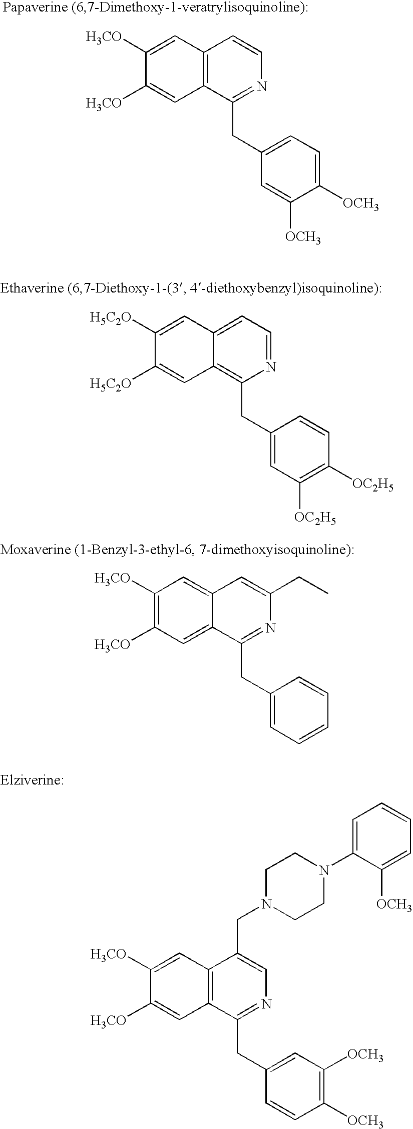

All the above-mentioned active substances can be used as a free base or as a pharmacologically compatible salt thereof, for example as a hydrochloride, sulfate, amidosulfate, etc. In that respect the active substances can be present in the form of acid addition salts.

For example the following salts have proven to be suitable: papaverine hydrochloride, papaverine sulfate, ethaverine hydrochloride, ethaverine amidosulfate, moxaverine hydrochloride, elziverine hydrochloride. It will be appreciated that it is also possible to use other pharmacologically compatible salts of the above-mentioned active substances or mixtures thereof.

Moxaverine and moxaverine hydrochloride respectively have proven to be highly suitable. The active substance moxaverine extremely advantageously has an elevated level of effectiveness and reduced toxicity, in relation to papaverine.

In animal testing it was surprisingly found that the papaverine-like vasodilators are very well compatible for the eye upon topical application to the surface of the eye.

In a compatibility study 50 μl of a moxaverine hydrochloride-bearing aqueous solution with a pH of 3.0-3.5 was applied to the right eye of twelve albino rabbits in each case over a period of 28 days with four applications per working day and two applications per weekend day in a single dose. The left eye served in each case as a control. Evaluation of the eye reaction was effected in accordance with the Table of EU Directive 92/69/EEC, Appendix, method B.5.

No rabbit exhibited swelling of the lids or any reddening of the conjunctiva. No changes to the iris or clouding of the cornea were observed in any of the twelve albino rabbits.

The papaverine-like vasodilators, preferably papaverine, ethaverine, moxaverine, elziverine, their pharmacologically compatible salts and mixtures thereof have a locally relaxing action on the blood vessels of the eye after topical application to the surface of the eye. This novel ophthalmological use of the above-mentioned active substances accordingly avoids the action on all blood vessels of the organism, which is unwanted in regard to systemic administration of vasodilators. The blood vessels of the eye are selectively dilated and accordingly blood circulation of the eye is improved.

The dilation of the blood vessels of the eye provides for an improvement in the microcirculation and thus an improved supply to the eye with nutrients and oxygen by way of the blood.

The use according to the invention extremely advantageously permits the treatment of ophthalmological dysfunctions which are linked to circulatory disturbances of the eye or which are to be attributed to circulatory disturbances of the eye.

In accordance with a preferred development the ophthalmological dysfunctions are selected from the group which consists of glaucoma, and ophthalmological dysfunctions linked to diabetes, for example neovascularisation glaucoma, hemorrhagic glaucoma or diabetic retinopathy.

In accordance with a further preferred embodiment the pharmaceutical composition is to be applied topically to the eye.

In accordance with a preferred development the pharmaceutical composition is in the form of eye drops, eye ointment, eye spray, eye tablet, gel, suspension, emulsion, powder or granules.

To produce the eye drops and the eye spray the active substances can be dissolved or suspended in buffer solutions which are usually employed such as for example phosphate buffer, acetate buffer, acetate borate buffer, borate buffer, citrate buffer etc.

It has surprisingly been found that the active substances do not have to be present in dissolved or completely dissolved form for application to the eye. Rather it is sufficient if the active substances are in suspension. The active substances are absorbed by the eye from the suspension applied to the surface of the eye.

In production of eye ointments the active substances can be formulated in usual ointment bases, for example in hydrocarbon gels with or without emulsifier additive, such as for example cholesterol, wool wax, wool wax alcohols, cetanol and so forth.

In accordance with a further preferred embodiment the pharmaceutical composition additionally includes a viscosity regulator.

Preferably the viscosity regulator is selected from the group which consists of chondroitin sulfate, polyacrylamide, polyacrylic acid, polyacrylic resins, polyethylene glycol, cellulose derivatives, polyvinyl alcohol, polyvinyl pyrrolidone, hyaluronic acid, hyaluronates and mixtures thereof.

Herein the term viscosity regulator is used to denote substances which are pharmacologically compatible and have a viscosity-increasing action. Preferably the viscosity regulator has a viscoelastic behaviour.

The viscosity-enhancing action extremely advantageously provides that the pharmaceutical composition applied to the surface of the eye has an increased residence time and does not flow away from the surface of the eye again.

A very suitable viscosity regulator is hyaluronic acid or salts thereof. For example potassium, sodium, calcium and/or magnesium hyaluronates can be used as salts of hyaluronic acid.

Preferably the hyaluronate is sodium hyaluronate.

Hyaluronic acid or hyaluronate involve a structural similarity with mucin, the lowermost layer of the triple-layer tear film, and provide for optimum wetting of the cornea and conjunctiva epithelia.

The hyaluronic acid and/or the hyaluronate accordingly imitates the mucous phase of the tear film and thus prolongs the residence time of the applied active substance on the eye as the viscosity counteracts the active substance from flowing away.

The non-Newtonian flow behaviour of the hyaluronic acid or the hyaluronates provides a property which is excellent in terms of use on the eye, namely that the viscosity decreases with increasing shearing speed. After the application of a composition with a papaverine-like active substance and a viscosity regulator, preferably hyaluronic acid or hyaluronate, to the cornea of the eye, a shearing stress is applied to the pharmaceutical composition by virtue of the eyelid blink whereby the initially increased viscosity is reduced. The eyelid blink reduces the viscosity so that a uniform film is produced on the surface of the eye. The viscosity increases after the blink so that the film adheres well to the surface of the eye. There is the very great advantage here that no impairment of sight is involved when using hyaluronic acid or salts thereof as a viscosity regulator.

Preferably the hyaluronic acid and/or the hyaluronate is of a molecular weight which is in a range of between about 50,000 and about 10,000,000 Daltons, preferably between about 250,000 and about 5,000,000 Daltons, further preferably between about 500,000 and 4,000,000 Daltons, still further preferably between about 1,500,000 and 3,500,000 Daltons.

The present invention further concerns a pharmaceutical composition which includes papaverine-like vasodilator and pharmacologically compatible viscosity regulator.

In accordance with a preferred development the papaverine-like vasodilator is selected from the group which consists of papaverine, ethaverine, moxaverine, elziverine, their pharmacologically compatible salts and mixtures thereof.

In a further preferred feature the viscosity regulator is selected from the group which consists of chondroitin sulfate, polyacrylamide, polyacrylic acid, polyacrylic resins, polyethylene glycol, cellulose derivatives, polyvinyl alcohol, polyvinyl pyrrolidone, hyaluronic acid, hyaluronate and mixtures thereof.

In accordance with a further development of the present invention the pharmaceutical composition is in the form of eye drops, eye ointment, eye spray, eye tablet, gel, suspension, emulsion, powder or granules.

If the pharmaceutical composition is in dry form, for example in the form of powder or granules, it can be dissolved or suspended in a liquid carrier, for example a sterile buffer solution, immediately prior to application to the surface of the eye, and it can then be applied.

In other respects reference is made to the foregoing information relating to use according to the invention of papaverine-like vasodilator, which correspondingly applies in regard to the pharmaceutical composition according to the invention.

The dosage of the papaverine-like vasodilators depends on the individual circumstances of the syndrome to which the therapy is to be applied and the person requiring the therapy. Insofar as the blood circulatory disturbances in the eye are detected at a very early time the dosage is less than at an advanced stage of the disease.

The amount of papaverine-like vasodilator contained in a unit dosage to be administered to the eye can be between 0.001 mg/ml and 100 mg/ml, preferably between 0.1 mg/ml and 50 mg/ml, further preferably between about 0.5 mg/ml and 10 mg/ml.

Preferably the active substance is in the form of an acid addition salt. Acid addition salts are generally good in terms of water solubility or better than the free base. Accordingly the above-mentioned liquid formulations such as drops, spray etc can be produced with an aqueous buffer solution.

In accordance with a preferred embodiment moxaverine hydrochloride is used. The moxaverine hydrochloride is preferably used as a solution in aqueous buffer solution with a pH of 3-3.5. After application of the aqueous solution to the surface of the cornea of the eye the active substance moxaverine hydrochloride is rapidly absorbed by the eye. Moxaverine hydrochloride however can also be used in the form of an aqueous suspension, in which case the active substance is only partially dissolved. Preferably moxaverine hydrochloride is used in a completely dissolved form in slightly acid or neutral solution, preferably with a pH of 3-7.5.

The active substance moxaverine is highly compatible even in a high dosage for human beings. Side effects of the active substance moxaverine are hitherto unknown. No interactions with other agents are also known. Thus, the use of moxaverine, besides further active substances, is possible in regard to therapy of ophthalmological dysfunctions.

In an extremely advantageous fashion papaverine-like vasodilators, preferably the active substance moxaverine, not only cause vasodilation of the blood vessels in the eye. Papaverine-like vasodilators also have an advantageous hemorheological action, that is to say they improve the flowability of the blood.

Accordingly the active substances employed in the use according to the invention and in the production of the pharmaceutical composition according to the invention produce two physiological effects which are extremely advantageous in terms of therapy for blood circulatory disturbances of the eye. On the one hand vessel dilation occurs, that is to say, a reduction in the flow resistance of the blood vessel. On the other hand the flow properties of the blood are improved. That synergistic effect affords a significant improvement in blood circulation of the eye.

The active substance moxaverine has proven to be highly suitable in that respect.

The deformability of erythrocyte membranes which are stiffened by stress, for example hypoxia, hyperosmolarity and lactacidosis is normalised under the action of moxaverine. The induced improvement in deformability of the erythrocytes, besides vessel dilation, results in improved blood circulation of the eye and thus an alleviation of the dysfunctions caused by the circulatory disturbance.

The example hereinafter shows, by reference to the active substance moxaverine hydrochloride, that, upon topical application of a moxaverine-bearing solution to the surface of the eye—in comparison with a systemic application—the active substance moxaverine is absorbed by the eye at high levels of concentration and on a long-lasting basis.

This example is only provided to further illustrate the present invention. The invention is in no way to be viewed as being limited to this embodiment thereof which is given by way of example.

### EXAMPLE

1. Manufacture of ¹⁴C-Marked Moxaverine Solution

19.03 g of water-free citric acid was dissolved in 200 ml of 1 M caustic soda solution. Demineralised water was then added to that solution, to 1000 ml. 100.75 ml of that stock solution was mixed with 0.1 M HCl in order to give 250 ml of buffer solution with a pH of 3.

4.41 mg of moxaverine hydrochloride was filled up ad 10 ml with the foregoing buffer solution and dissolved. ¹⁴C-marked moxaverine was added to that moxaverine-bearing solution until an activity of 100 μCi/ml was achieved. The solution was adjusted to be iso-osmolar with the addition of NaCl.

2. Animal-Experimental Investigation

The experimental investigations were carried out on rabbits (male, pigmented striped Dutch rabbits, which can be obtained from Irish Farm, Norco, Calif., USA), the moxaverine-bearing solution being administered topically and also systemically. The absorption of moxaverine was determined after 30 minutes and after 120 minutes. One rabbit was used for each moment in time and for each kind of application.

2.1 Ocular Application

50 μl of the ¹⁴C-marked moxaverine solution described in 1. was trickled with a 100 μl pipette into each of the two rabbit eyes.

2.2 Systemic Application

100 μl of the ¹⁴C-marked moxaverine solution described in 1. was injected after disinfection of the ear intravenously into the lateral vein of the rabbit ear.

2.3 Sample Preparation

After 30 and 120 minutes respectively blood was taken from each rabbit from the central artery of the ear, using a heparinised syringe. The heparin prevented coagulation of the removed blood.

After the blood was taken the rabbits were each killed by the administration of an overdose of Eutha-6 CII-pentobarbital sodium solution (this can be obtained from Western Medical Supply Inc, Arcadia, Calif., USA) into the lateral vein of the rabbit ear. The rabbit eyes were then each prepared as follows:

In each case a liquid sample of the aqueous humor was taken from the first eye, and a liquid sample from the vitreous humor, using a syringe.

The second eye was cut open for the purposes of obtaining tissue. Using a scalpel of size 10, a cut was made around the entire eye at a distance of about 1 cm from the outer limbus of the eye. The skin was lifted off in order to get into the orbit. The tissue between the eyeball and the orbit was severed. The four eye muscles were severed with surgical scissors and the eyeball was removed from the orbit. The optic nerve was severed. The eye was then washed in 1× phosphate-buffered saline (this can be obtained from Gibco, Grand Island, N.Y., USA) in order to remove blood and hair.

The skin and the flesh were removed from the rear of the eye using tweezers, scissors and scalpel. The flesh or skin around the eye was cut off along the limbus between the skin and the conjunctiva. The conjunctiva was then removed. The cornea was removed by using a scalpel of size 11 to cut along the sclera. The iris and the ciliary process were removed together with the cornea and freed of tissue. The crystalline lens could then be removed with a pair of tweezers. The rest of the vitreous humor was expelled and the outer layers of the eyeball were cut eight times in order to lay them flat.

All pieces of tissue were transferred into scintillation tubes which each contained 1 ml of tissue solubiliser (0.5 N caustic soda solution, 15% by volume of Triton X-100 in Ringer solution with bicarbonate). The tissue samples were irradiated with ultrasound for between 12 and 18 hours in order to destroy the tissue.

After the sample tubes were weighed out 5 ml of scintillation cocktail Econo-Safe® (this can be obtained from Research Products Intl. Corp. Mount Prospect, Ill., USA) was added and then the radioactive radiation was measured in a scintillation counter from Beckman in accordance with the manufacturers' specifications.

3. Results

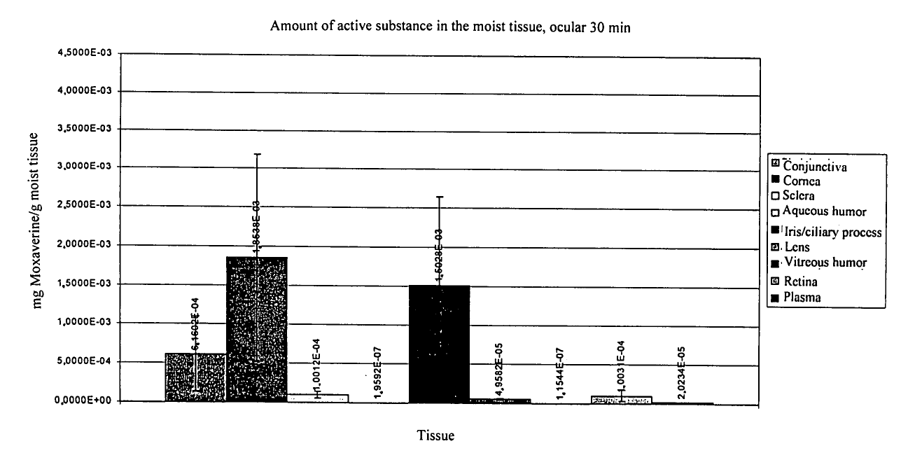

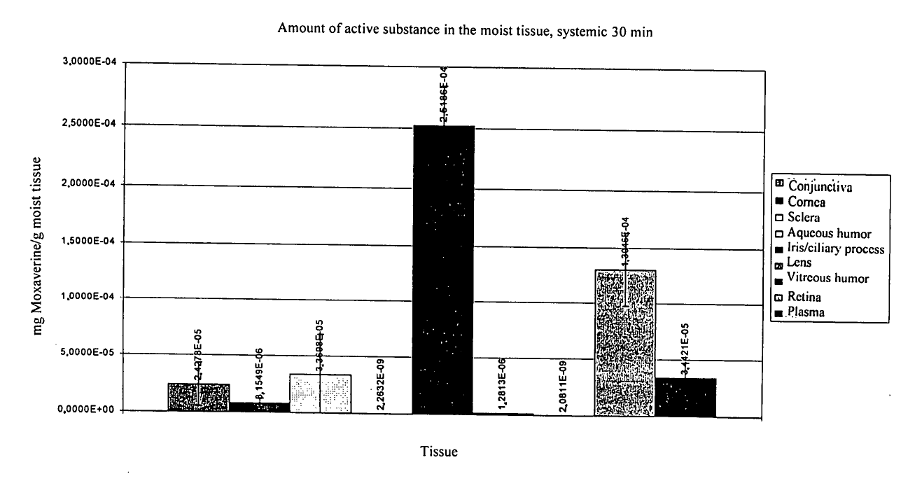

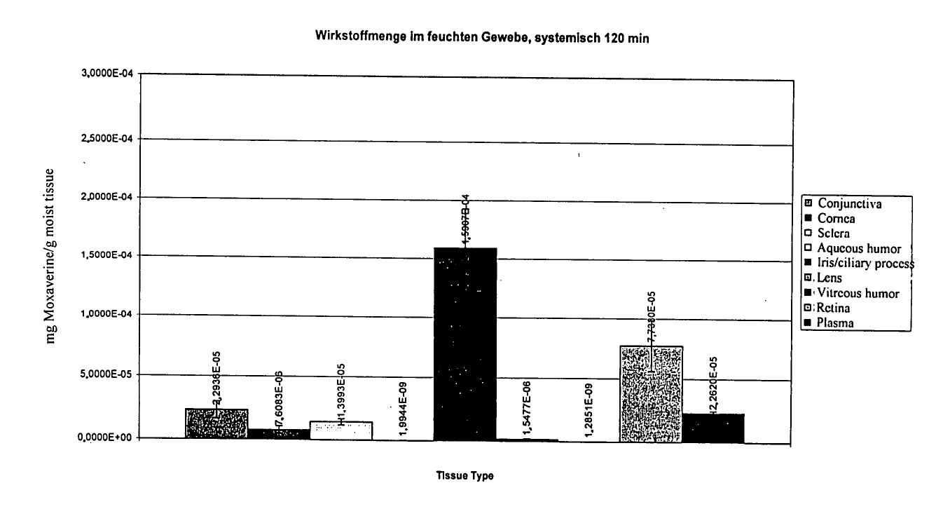

The results are set forth in Tables 1 through 4 and shown in graph form in FIGS. 1 through 4. The information in the Tables is in [mg of moxaverine/g of moist tissue].

TABLE 1
Amount of active substance in the moist tissue with ocular
application after 30 minutes

| Tissue | Experiment 1 | Experiment 2 | Experiment 3 | Mean value | SD |
|---|---|---|---|---|---|
| Conjunctiva | 5.4510E−05 | 8.7953E−04 | 9.1403E−04 | 6.1602E−04 | 4.8659E−04 |
| Cornea | 5.4061E−04 | 3.2044E−03 | 1.8164E−03 | 1.8538E−03 | 1.3323E−03 |
| Sclera | 4.7303E−05 | 1.3593E−04 | 1.1713E−04 | 1.0012E−04 | 4.6698E−05 |
| Aqueous | 1.4923E−07 | 2.6321E−07 | 1.7533E−07 | 1.9529E−07 | 5.9715E−08 |
| humor | | | | | |
| Iris/ciliary | 6.2428E−04 | 1.0949E−03 | 2.7891E−03 | 1.5028E−03 | 1.1386E−03 |
| process | | | | | |
| Lens | 4.6171E−05 | 6.1860E−05 | 4.0715E−05 | 4.9582E−05 | 1.0977E−05 |
| Vitreous | 5.6527E−08 | 1.4047E−09 | 2.8840E−07 | 1.1544E−07 | 1.5230E−07 |
| humor | | | | | |
| Retina | 1.7239E−05 | 1.3276E−04 | 1.5094E−04 | 1.0031E−04 | 7.2516E−05 |
| Plasma | 2.3741E−05 | 1.8259E−05 | 1.8702E−05 | 2.0234E−05 | 3.0452E−06 |

TABLE 2
Amount of active substance in the moist tissue with ocular
application after 120 minutes

| Tissue | Experiment 1 | Experiment 2 | Experiment 3 | Mean value | SD |
|---|---|---|---|---|---|
| Conjunctiva | 1.0247E−04 | 6.0912E−04 | 4.0667E−04 | 3.7275E−04 | 2.5502E−04 |
| Cornea | 3.0586E−04 | 1.6630E−03 | 1.3481E−03 | 1.1057E−03 | 7.1031E−04 |
| Sclera | 4.6987E−05 | 1.2822E−04 | 1.3469E−04 | 1.0330E−04 | 4.8875E−05 |
| Aqueous | N/A | 5.8323E−08 | 1.2851E−07 | 9.3417E−08 | 4.9630E−08 |
| humor | | | | | |
| Iris/ciliary | 1.3547E−03 | 3.5424E−03 | 4.0576E−03 | 2.9849E−03 | 1.4351E−03 |
| process | | | | | |
| Lens | 4.2292E−05 | 3.6044E−05 | 9.2004E−05 | 5.6780E−05 | 3.0664E−05 |
| Vitreous | 2.7133E−08 | 7.02373E−10 | 9.9991E−10 | 9.6118E−09 | 1.5175E−08 |
| humor | | | | | |
| Retina | 1.8933E−04 | 1.4755E−04 | 9.5039E−05 | 1.4397E−04 | 4.7247E−05 |
| Plasma | 1.6619E−05 | 1.4435E−05 | 7.6179E−06 | 1.2891E−05 | 4.6951E−06 |

TABLE 3
Amount of active substance in the moist tissue with systemic
application after 30 minutes

| Tissue | Experiment 1 | Experiment 2 | Experiment 3 | Mean value | SD |
|---|---|---|---|---|---|
| Conjunctiva | 1.0655E−05 | 3.7901E−05 | | 2.4278E−05 | 1.9266E−05 |
| Cornea | 5.2937E−06 | 1.1016E−05 | | 8.1549E−06 | 4.0463E−06 |
| Sclera | 8.5411E−06 | 5.8944E−05 | | 3.3698E−05 | 3.5704E−05 |
| Aqueous | 2.0291E−09 | 2.4973E−09 | | 2.2632E−09 | 3.3107E−10 |
| humor | | | | | |
| Iris/ciliary | 2.2898E−04 | 2.7473E−04 | | 2.5186E−04 | 3.2350E−05 |
| process | | | | | |
| Lens | 9.5659E−07 | 1.6061E−06 | | 1.2813E−06 | 4.5927E−07 |
| Vitreous | 2.6534E−09 | 1.5088E−09 | | 2.0811E−09 | 8.0935E−10 |
| humor | | | | | |
| Retina | 1.0666E−04 | 1.5425E−04 | | 1.3046E−04 | 3.3651E−05 |
| Plasma | 2.8375E−05 | 4.9032E−05 | 2.5855E−05 | 3.4421E−05 | 1.2716E−05 |

TABLE 4
Amount of active substance in the moist tissue with systemic
application after 120 minutes

| Tissue | Experiment 1 | Experiment 2 | Experiment 3 | Mean value | SD |
|---|---|---|---|---|---|
| Conjunctiva | 2.7539E−05 | 1.5261E−05 | 2.6009E−05 | 2.2936E−05 | 6.6909E−06 |
| Cornea | 1.0406E−05 | 4.6148E−06 | 7.8042E−06 | 7.6083E−06 | 2.9006E−06 |
| Sclera | 1.4230E−05 | 1.1476E−05 | 1.6272E−05 | 1.3993E−05 | 2.4068E−06 |
| Aqueous | 2.3412E−09 | 1.8210E−09 | 1.8210E−09 | 1.9944E−09 | 3.0034E−10 |
| humor | | | | | |
| Iris/ciliary | 1.2211E−04 | 1.6708E−04 | 1.8802E−04 | 1.5907E−04 | 3.3677E−05 |
| process | | | | | |
| Lens | 1.3706E−06 | 2.0870E−06 | 1.1854E−06 | 1.5477E−06 | 4.7617E−07 |
| Vitreous | 1.5608E−09 | 9.6772E−10 | 1.3267E−09 | 1.2851E−09 | 2.9872E−10 |
| humor | | | | | |
| Retina | 9.5067E−05 | 8.3068E−05 | 5.3855E−05 | 7.7330E−05 | 2.1197E−05 |
| Plasma | 2.4420E−05 | 2.1190E−05 | 2.2250E−05 | 2.2620E−05 | 1.6465E−06 |

The respective averaged measurement values from Tables 1 through 4 are shown in FIGS. 1 through 4.

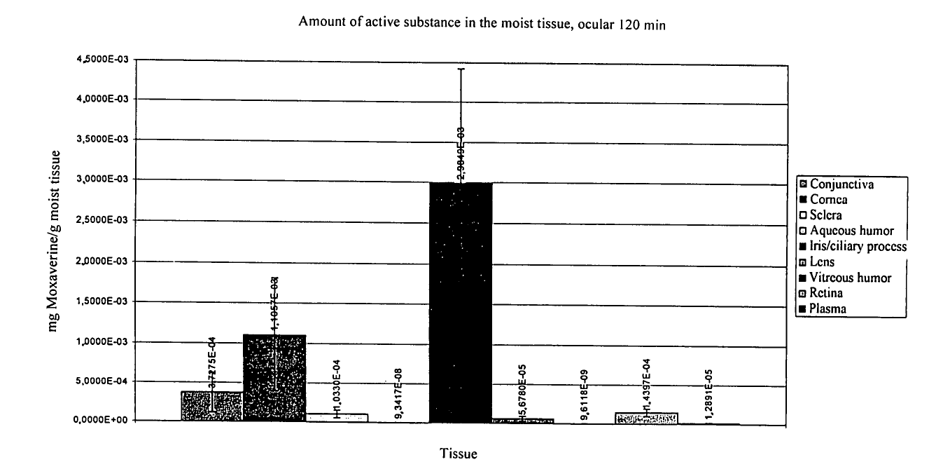

It can be clearly seen from FIGS. 1 and 2 that the active substance moxaverine upon ocular application is increased in concentration in the conjunctiva, the cornea, the iris, the ciliary process as well as the retina. After 120 minutes the concentration in the conjunctiva and the cornea has decreased whereas the active substance concentration in the iris and the ciliary process has almost doubled. In the retina the concentration of moxaverine after 120 minutes has increased by about 50%, compared to the time after 30 minutes.

The results show that the active substance moxaverine is very well absorbed by the eye after topical application to the surface thereof.

With systemic administration of moxaverine, after 30 minutes a level of concentration is reached in the retina, which is slightly above that when topical administration was involved.

With systemic application, the concentration of moxaverine in the iris and the ciliary process respectively at the time of 30 minutes is less approximately by a factor of 6 than when topical application was involved. After 120 minutes the moxaverine concentration in the iris and the ciliary process respectively after systemic application is less approximately by a factor of 18 than in the case of topical application.

It is also to be noted that the concentration of moxaverine in the iris and the ciliary process at the time of 120 minutes is only about 63% of the concentration after 30 minutes with systemic application. In contrast, in the case of topical application, the concentration of moxaverine in the iris and the ciliary process respectively increases from the time of 30 minutes to the time of 120 minutes approximately by a factor of 2.

With systemic application, the concentration of moxaverine in the retina also decreases by a factor of 1.6 from 30 to 120 minutes whereas with topical application it increases by a factor of 1.4 in the same period.

These results clearly show that, with the same application amount the active substance moxaverine is better absorbed by the eye with topical application and exhibits a markedly longer biological availability in the eye. Accordingly topical application of moxaverine to the eye permits markedly longer and more stable therapy than is possible with the systemic application of moxaverine.

The advantageous topical administration of papaverine-like vasodilator was shown in the foregoing example by reference to moxaverine.

1. A method of treating an ophthalmological dysfunction which is linked to circulatory disturbances of the eye or which can are to be attributed to circulatory disturbances of the eye, comprising administering a pharmaceutical composition comprising a papaverine-like vasodialator, wherein said pharmaceutical composition is administered topically to said eye.

2. The method according to claim 1 wherein the papaverine-like vasodilator is selected from the group consisting of papaverine, ethaverine, moxaverine, elziverine, their pharmacologically compatible salts and mixtures thereof.

3. The method according to claim 1 wherein the ophthalmological dysfunction is selected from the group consisting of glaucoma and an ophthalmological dysfunction linked to diabetes.

4. The method according to claim 1 wherein the pharmaceutical composition is in a form selected from the group consisting of eye drops, eye ointments, eye spray, eye tablet, gel, suspension, emulsion, powder and granules.

5. The method according to claim 1 wherein the pharmaceutical composition includes a viscosity regulator, wherein the viscosity regulator has a viscosity-increasing action.

6. The method according to claim 5 wherein the viscosity regulator is selected from the group consisting of chondroitin sulfate, polyacrylamide, polyacrylic acid, polyacrylic resins, polyethylene glycol, cellulose derivatives, polyvinyl alcohol, polyvinyl pyrrolidone, hyaluronic acid, hyaluronates and mixtures thereof.

7. A pharmaceutical composition which includes a papaverine-like vasodilator and a pharmacologically compatible viscosity regulator, wherein the papaverine-like vasodilator is selected from the group consisting of moxaverine, its pharmacologically compatible salts and mixtures thereof, and wherein the viscosity regulator is selected from the group consisting of chondroitin sulfate, polyacrylamide, polyacrylic acid, polyacrylic resins, polyethylene glycol, cellulose derivatives, polyvinyl alcohol, polyvinyl pyrrolidone, hyaluronic acid, hyaluronates and mixtures thereof.

8. A pharmaceutical composition as set forth in claim 7 wherein the pharmaceutical composition is in a form selected from the group consisting of eye drops, eye ointments, eye spray, eye tablet, gel, suspension, emulsion, powder and granules.

9. The method according to claim 3 wherein said ophthalmological dysfunction linked to diabetes is selected from the group consisting of neovascularisation glaucoma and diabetic retinopathy.
